(19) United States
(12) Patent Application Publication
(10) Pub. No.: US 2026/0115114 A1
(43) Pub. Date: Apr. 30, 2026

(54) COSMETIC COMPOSITION THAT RESISTS PILLING

(71) Applicant: L'OREAL, Paris (FR)

(72) Inventor: Karoline Elise TUELL, Rahway, NJ (US)

(73) Assignee: L'OREAL, Paris (FR)

(21) Appl. No.: 18/930,542

(22) Filed: Oct. 29, 2024

(51) Int. Cl. A61K 8/368; A61K 8/365; A61K 8/44; A61Q 19/00

(52) U.S. Cl. CPC A61K 8/368; A61K 8/365; A61K 8/445; A61Q 19/007; A61K 2800/48

(57) ABSTRACT

A cosmetic moisturizing peel cream composition includes at least one hydroxy acid, at least one surfactant, at least one polymer thickener, at least one emollient, at least one water based solvent, wherein the ratio between (emollient):(surfactant) is from about 1:1 to about 4:1. The moisturizing peel cream compositions is thick, stable, and resistant to pilling and has a pH of about 3.5 to about 6.3.

### FIELD OF THE INVENTION

The instant disclosure is generally directed to a pilling-resistant cosmetic composition and method for manufacturing the same for providing benefits to the skin of a user.

### BACKGROUND OF THE INVENTION

There are a variety of skin conditions that benefit from leave-on cosmetic compositions that may be applied on the skin for any of a variety of possible benefits. In some examples, it is challenging to formulate skin care cosmetics that include sufficiently high amounts of actives to confer desired benefits. Certain actives are challenging to formulate due to their impact on formula stability and sensory experience. Compositions with high concentration of acids/salts must be either liquid or serum texture; or if thick cream texture is desired (i.e., for luxe skincare), a high concentration of thickener must be used to build viscosity and stabilize. A high concentration of thickener can lead to pilling and other sensory drawbacks.

Accordingly, there is a need in the art for a cosmetic composition that can deliver high amounts of actives (especially acids and/or functional electrolytes) while offering a thick, creamy and luxurious texture without piling on skin during application, after drying down, or during combined use (layering) with other cosmetic products.

### BRIEF SUMMARY OF THE INVENTION

The summary is provided to introduce a selection of concepts in a simplified form that are further described below in the detailed description of the invention. This summary is not intended to identify key features of the claimed subject matter, nor is it intended to be used as an aid in determining the scope of the claimed subject matter.

The instant case relates to moisturizing peel cream compositions having a high amount of acids that achieve a thick, creamy and luxurious texture without piling on skin during application, after drying down, or during combined use (layering) with other cosmetic products such as toner, serum and/or eye cream. For example, skin care compositions with high amounts of skin exfoliating acids or functional electrolytes are particularly challenging to formulate due to the negative impact of these ingredients on formula stability and sensory. Many emulsifiers and viscosity modifiers known to those of ordinary skill in the art are incompatible with or have low efficiency in the presence of high amounts of acids and electrolytes or in low pH environments.

To circumvent the formulation challenges associated with incorporation of high amounts of these actives, a cosmetic galenic may be selected wherein optimal efficiency of conventional emulsifiers and thickeners is not paramount to the stability of the composition—for example, in toner, serum, or loose lotion formats. However, it is well known that consumers of high-end, luxury cosmetics desire the sensory experience of a thick and luxurious cream emulsion galenic, wherein nourishing emollients are incorporated into the composition to impart additional sensorial and skin benefits such as comfort, moisturization, and soothing. In order to compensate for the low efficiency of viscosity modifiers in the presence of high amounts of active ingredients, one must add a high amount of viscosity modifiers (such as polymer thickeners) in order to achieve a thick cream texture that appeals to the luxury consumer. This approach often leads to sensorial drawbacks such as pilling and sticky skin finish. The invention described herein overcomes the common tradeoff between a thick, stable texture and a good sensorial experience without pilling for compositions containing a high amount of skin exfoliating acids.

The moisturizing peel cream compositions are stable, showing no phase separation or significant changes in odor, color, texture, or pH with time and elevated temperature.

The moisturizing peel creams typically include:
from up to about 20 wt. % of at least one hydroxy acid;
from about at least 6 wt. % of at least one surfactant;
at least 0.2% of one polymer thickener;
at least one emollient;
at least one water-based solvent,
water;
wherein the ratio between (emollient):(surfactant) is from about 1:1 to about 4:1;
wherein the composition has a complex viscosity above 200 Pa·S, is stable, resistant to pilling and has a pH is of about 3.5 to about 6.3; and
wherein the weight percentages are based on the total weight of the composition.

In various embodiments, the moisturizing peel creams contain at least one hydroxy acid is chosen from alpha-, poly-, beta-hydroxy acids, and combinations thereof.

In some embodiments, when the moisturizing peel creams contain an alpha hydroxy acid, it is present at less than about 8 wt. % based on the total weight of composition. In some embodiments, the alpha-hydroxy acid is chosen from glycolic acid, lactic acid, tartaric acid, mandelic acid, citric acid, glycolic citric acid, mandelic acid, tartaric acid, kojic acid, malic acid, ester derivatives thereof and combinations thereof. In various embodiments, the beta-hydroxy acid is chosen from salicylic acid, betaine salicylate, tropic acid and mixture thereof. In some embodiments, the poly-hydroxy acid is chosen from gluconolactone, lactobionic acid, galactose, and mixture thereof. In various embodiments, the moisturizing peel cream is free of beta-hydroxy acid. In various embodiments, the moisturizing peel cream is free of poly hydroxy acid.

In some embodiments, the moisturizing peel cream further comprises at least one additional hydroxy acid chosen from alpha-, poly-, and beta-hydroxy acids, and combinations thereof.

In various embodiments, the moisturizing peel cream comprises at least one surfactant chosen from fatty alcohol, cetearyl alcohol, stearic acid, polysorbate 20, ceteareth-20, PEG-100 stearate, polyglyceryl-10 stearate, glyceryl stearate, sorbitan stearate, and combinations thereof. In some embodiments, the at least one surfactant is present from at least about 6.0 wt. % based on the total weight of the composition. In some embodiments, the at least one surfactant is present from at least about 6.5 wt. % based on the total weight of the composition. In one embodiment, the at least one surfactant is present from about 6.1 to about 15 wt. % based on the total weight of the composition.

In various embodiments, the moisturizing peel cream comprises at least one polymer thickener chosen from xanthan gum, sclerotium gum, hydroxypropyl starch phosphate, cetyl hydroxyethyl cellulose, carbohydrate based gellifying agents, organic thickeners and mixture thereof. In some embodiments, the amount of polymer thickeners is up to about 0.45 wt. % based on the total weight of the composition.

In various embodiments, the moisturizing peel cream comprises at least one emollient chosen from plant butters, oils, and combinations thereof. In some embodiments, the at least emollient is chosen from Butyrospermum parkii butter, Theobroma cacao seed butter, Mangifera indica (mango) seed butter, Astrocaryum murumuru seed butter, Simmondsia chinensis (jojoba) butter, caprylic/capric triglyceride, coco-caprylate/caprate, C12-15 alkyl benzoate, sunflower seed oil, olive oil, coconut oil, C15-19 alkane, C9-12 alkane, squalane, isopropyl myristate, and combinations thereof.

In various embodiments, the moisturizing peel cream comprises at least one water-based solvents is chosen from glycerin, propylene glycol, propanediol, butylene glycol, pentylene glycol, sorbitol, xylitol and mixture thereof. In some embodiments, the at least one water-based solvent is present from about 5 wt. % to about 10 wt. % based on the total weight of the composition.

In various embodiments, the moisturizing peel cream comprises water. In one embodiment, the water is present from about 30 wt. % to about 80 wt. % based on the total weight of the composition.

In various embodiments, the moisturizing peel cream has a complex viscosity above 200 Pa·S.

In various embodiments, the moisturizing peel cream further comprises at least one texturizer. In some embodiments, the at least one texturizer is chosen from silica, cellulose, boron nitride, mica, Hordeum vulgare seed flour, Aluminum Starch Octenylsuccinate and combination thereof.

In various embodiments, the moisturizing peel cream is free or essentially free from silicones.

Another aspect of the instant disclosure relates to method for treating skin. The methods include applying the cosmetic composition according to the instant disclosure to the skin, in particular the skin of the face.

Other features and advantages of the present invention will be apparent from the following more detailed description, by way of example, the principles of the invention.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### DETAILED DESCRIPTION OF THE INVENTION

As used herein, the term “enhancing” of skin refers to effects on skin as a result of use of the inventive moisturizing peel cream that can include one or more of improved or reduced pore appearance, improved visual skin smoothness, improved skin brightness, and reduced fine lines and wrinkles.

The term “silicone-free” as used herein means containing less than 5%, such as less than 3%, such as less than 1% by weight, based on the total active weight of the composition, of silicones, such as silicone polymers, for example selected from dimethicone, cyclopentasiloxane, cyclohexasiloxane and other silicone oils, and silicone elastomers.

The term “pilling” as used herein occurs when a cosmetic formula aggregates on the skin in response to friction (i.e., rubbing) causing the formation of small globs, strings, flakes, or mounds on the skin. Pilling may occur during product application or after the product has dried down. Pilling may be exacerbated when multiple cosmetic products are layers atop each other.

The term “polymer thickener” as used herein means that the thickener can dissolve or swell in the aqueous phase, resulting in an increase in the viscosity of the composition and/or stabilization of an emulsions system.

The term “texturizer” or “filler” as used herein means that the raw material does not dissolve but it modifies the texture of the composition or the skin finish.

As described and exemplified in various embodiments herein, the inventors have created a pilling-resistant moisturizing peel cream in the form of an oil-in-water emulsion comprising:
from up to about 20 wt. % of at least one hydroxy acid;
from about at least 6 wt. % of at least one surfactant;
at least 0.2% of one polymer thickener;
at least one emollient;
at least one water-based solvent,
water;
wherein the ratio between (emollient):(surfactant) is from about 1:1 to about 4:1;
wherein the composition has a complex viscosity above 200 Pa·S, is stable, resistant to pilling and has a pH is of about 3.5 to about 6.3; and
wherein the weight percentages are based on the total weight of the composition.

### Hydroxy Acid

In some embodiments, the moisturizing peel cream has a pH of about 3.5 to about 6.3. The moisturizing peel cream is thick, stable and resistant to pilling. The emulsion contains at least one hydroxy acid chosen from alpha-, poly-, beta-hydroxy acids, and combinations thereof.

### Alpha Hydroxy Acid

In accordance with the various embodiments, the moisturizing peel cream comprises at least one alpha hydroxy acid.

Suitable alpha hydroxy acids include glycolic acid, lactic acid, tartaric acid, mandelic acid, citric acid, ester derivatives thereof and combinations thereof. Exemplary ester derivatives include ester compounds of glycolic acid, lactic acid, such as methyl lactate, ethyl lactate, butyl lactate and, similarly, ester compounds of glycolic acid, tartaric acid, mandelic acid, citric acid. One particularly suitable alpha hydroxy acid is glycolic acid. In some embodiments, lactic acid, or 2-hydroxypropanoic acid, is provided to the chemical peel composition to provide enhanced exfoliation of the skin. In addition, lactic acid also boosts production of glycosaminoglycan (GAG) in the skin, improving the barrier function and moisturization of skin.

In some embodiment, the alpha hydroxy acid, if present, is less than about 8 wt. % based on the total weight of composition. In various embodiments, the alpha-hydroxy acid is chosen from glycolic acid, lactic acid, tartaric acid, mandelic acid, citric acid, glycolic citric acid, kojic acid, malic acid, ester derivatives thereof and combinations thereof.

The moisturizing peel cream includes a concentration of alpha hydroxy acid in a range from about at least about 3.0%, or from about 3.5% to about 8%, or from about 3.0% to about 8% or from about 3.5% to about 7.8%, or is about 6%, or is less than 8%, or any suitable combination, sub-combination, range, or sub-range thereof by weight, based on the weight of the moisturizing peel cream. One of ordinary skill in the art, however, will appreciate that other ranges are within the scope of the invention. In accordance with some embodiments, the amount of alpha hydroxy acid present is not more than about 8%.

Thus, any one of or a combination of alpha hydroxy acid is present, by weight, based on the total weight of the moisturizing peel cream, from about 3.0, 3.5, 4.0, 4.5, 5.0, 5.5, 6.0, 6.5, 7.0, 7.5, about 8.0 weight percent, including increments and ranges therein and there between.

### Beta Hydroxy Acid

In accordance with the various embodiments, the moisturizing peel cream may comprise at least one beta hydroxy acid. In some particular embodiments, the moisturizing peel cream may include a beta hydroxy acid comprising salicylic acid.

The term “beta-hydroxy acid” is understood to mean, according to the present invention, a carboxylic acid having a hydroxyl functional group and a carboxylic functional group separated by two carbon atoms. A beta hydroxy acid can be present in the moisturizing peel cream in the form of the free acid and/or in the form of one of its associated salts (salts with an organic base or an alkali metal, in particular), especially according to the final pH imposed on the moisturizing peel cream.

Suitable beta hydroxy acids include salicylic acid and derivatives thereof (including 5-n-octanoylsalicylic acid, salicylate, sodium salicylate, and willow extract), capryloyl salicylic acid, beta hydroxybutanoic acid, propionic acid, beta-hydroxy beata-methylbutyric acid, carnitine tropic acid, and trethocanic acid, and combinations of these.

In various embodiments, the beta-hydroxy acid is chosen from salicylic acid, betaine salicylate, tropic acid and mixture thereof.

And in some particular embodiments, the beta hydroxy acid in the moisturizing peel cream excludes at least one of salicylic acid and derivatives thereof (including 5-n-octanoylsalicylic acid, salicylate, sodium salicylate, and willow extract), capryloyl salicylic acid, beta hydroxybutanoic acid, propionic acid, beta-hydroxy beata-methylbutyric acid, carnitine tropic acid, and trethocanic acid.

The moisturizing peel cream may include a concentration of beta hydroxy acid in a range from about 0.1% up to and not more than about 2% of beta hydroxy acid, by weight, based on the total weight of the moisturizing peel cream. In some embodiments, the moisturizing peel cream includes up to and not more than about 2%, or about 1.9% of beta hydroxy acid. In some embodiments, the moisturizing peel cream includes up to and not more than about 1% of beta hydroxy acid. In accordance with some embodiments, the amount of beta hydroxy acid present is not more than about 0.40% to about 0.50%. In some embodiments, the moisturizing peel cream includes from about 0.1% to about 1% of beta hydroxy acid, or from about 0.2% to about 2.0%, or from about 0.1% to about 1.5%, or from about 0.2% to about 1.5%, or from about 0.3% to about 1.0%, or from about 0.35% to about 0.75%, or from about 0.4% to about 0.5%, or is about 0.45%, or any suitable combination, sub-combination, range, or sub-range thereof by weight, based on the weight of the moisturizing peel cream. One of ordinary skill in the art, however, will appreciate that other ranges are within the scope of the invention.

Thus, the at least one beta hydroxy acid, when present, is present in the moisturizing peel cream from about 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, to about 2.0 weight percent, including increments and all ranges and subranges therein and there between.

### Poly Hydroxy Acid

In accordance with the various embodiments, the moisturizing peel cream may comprise at least one poly hydroxy acid.

Suitable poly hydroxy acids include gluconolactone and derivatives thereof and combinations thereof. In various embodiments, the poly-hydroxy acid is selected from the group consisting of gluconolactone, lactobionic acid, galactose, and combination thereof.

The moisturizing peel cream may include a concentration of poly hydroxy acid in a range from about 3.0% to about 15%, or from about 3.5% to about 14% or from about 4.0% to about 13%, or from about 4.0% to about 10%, or from about 4.0% to about 9.0%, or is about 4.0%, or any suitable combination, sub-combination, range, or sub-range thereof by weight, based on the weight of the moisturizing peel cream. One of ordinary skill in the art, however, will appreciate that other ranges are within the scope of the invention. In accordance with some embodiments, the amount of poly hydroxy acid present is not more than about 10%.

Thus, any one of or a combination of poly hydroxy acid, when present, is present, by weight, based on the total weight of the moisturizing peel cream, from about 3.0, 4.0, 5.0, 6.0, 7.0, 8.0, 9.0, 10, 11, 12, 13, 14, to about 15 weight percent, including increments and ranges therein and there between.

In one embodiment, the moisturizing peel cream is free of beta-hydroxy acid.

In one embodiment, the moisturizing peel cream is free of poly hydroxy acid.

In various embodiments, the moisturizing peel cream further comprising at least one additional hydroxy acid selected from the group consisting of alph-, poly-, and beta-hydroxy acids, and combinations thereof.

### Surfactants

In accordance with the various embodiments, the moisturizing peel cream includes at least one surfactant.

In accordance with the various embodiments, the moisturizing peel cream includes at least one surfactant comprising one or a combination of surfactants, for example selected from the group consisting of fatty alcohol, cetearyl alcohol, stearic acid, polysorbate 20, ceteareth-20, PEG-100 stearate, polyglyceryl-10 stearate, glyceryl stearate, sorbitan stearate, and combinations thereof.

In some embodiments, the at least one surfactant is present from about at least 6 wt. % based on the total weight of the moisturizing peel cream. In some embodiments, the at least one surfactant is present from at least about 6.5 wt. % based on the total weight of the composition. In some embodiments, the at least one surfactant is present from about 6.5 to about 15 wt. % based on the total weight of the composition.

In accordance with the various embodiments, the amount of surfactant present in the composition may be from about 6% to about 15%, alternatively from about 6.5% to about 14%, alternatively from about 7% to about 15%, alternatively from about 7.5% to about 14%, alternatively from about 8% to about 13%, or any suitable combination, sub-combination, range, or sub-range thereof by weight, based on the weight of the composition. One of ordinary skill in the art, however, will appreciate that other ranges are within the scope of the invention.

In some embodiments, surfactants selected are sulfate free. In some particular embodiments, the blend of surfactants comprises two or more surfactants that are sulfate-free. The term “sulfate-free” as used herein means that sulfate has not been added as a component. In some embodiments, a composition is devoid of sulfate. Those of skill in the art will appreciate that sulfate may be present in a composition via its presence in one or more of the formulation components; thus, in some embodiments a composition may be “essentially sulfate-free” wherein sulfate is present at a concentration that does not exceed 5% by weight, and in some instances is present not more than 3% by weight, and in some instances is present not more than 1% by weight, based on the weight of the cleansing composition. Thus, the term “sulfate-free” means that the surfactant does not include sulfate.

In accordance with the various embodiments, each one of the surfactants in the blend of surfactants is present in the composition in an amount from about 0.5% to about 15%, or from about 1% to about 4%, or any suitable combination, sub-combination, range, or sub-range thereof by weight, based on the weight of the moisturizing peel cream. In some embodiments, a blend of surfactants is present in the composition in an amount from about 5% to about 8%, or any suitable combination, sub-combination, range, or sub-range thereof by weight, based on the weight of the moisturizing peel cream. One of ordinary skill in the art, however, will appreciate that other ranges are within the scope of the invention.

Thus, each one of the surfactants, alone or in combination, is present in an amount by weight, based on the total weight of the moisturizing peel cream, from about 0.5, 0.6, 0.7, 0.8, 0.9, 1, 2, 3, 4, 5, 6, 7, 8, 9, 10, 11, 12, 13, 14 to about 15 weight percent, including increments and ranges therein and there between.

### Polymer Thickener

In accordance with the various embodiments, the moisturizing peel cream includes at least one polymer thickener chosen from xanthan gum, sclerotium gum, hydroxypropyl starch phosphate, cetyl hydroxyethyl cellulose, carbohydrate based gellifying agents, organic thickeners and combination thereof.

In some embodiments, the total amount of the at least one polymer thickeners is up to about 0.45 wt. % based on the total weight of the composition. In various embodiments, the total amount of polymer thickeners is up to about 0.4 wt. % based on the total weight of the composition. In further embodiments, the total amount of polymer thickeners is up to about 0.35 wt. % based on the total weight of the composition.

The total amount of the at least one polymer thickeners in the composition will vary but is typically from about 0.20 to about 0.45 wt. % based on the total weight of the composition. In further embodiments, the composition includes about 0.25 to about 0.38 or any suitable combination, sub-combination, range, or sub-range thereof by weight, based on the weight of the moisturizing peel cream. One of ordinary skills in the art, however, will appreciate that other ranges are within the scope of the invention.

Thus, each one of the polymer thickeners, alone or in combination, is present in an amount by weight, based on the total weight of the moisturizing peel cream, from about, 0.20, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.40, 0.41, 0.42, 0.43, 0.44, or to about 0.45 weight percent, including increments and ranges therein and there between.

### Oils

In accordance with the various embodiments, the moisturizing peel cream includes one or more oils.

In some embodiments, the moisturizing peel cream includes at least one emollient chosen from plant butters, oils, and combinations thereof. Nonlimiting examples of emollients may include Butyrospermum parkii butter, Theobroma cacao seed butter, Mangifera indica (mango) seed butter, Astrocaryum murumuru seed butter, Simmondsia chinensis (jojoba) butter and combinations thereof.

In the various embodiments, the moisturizing peel cream includes at least one emollient selected from polar emollients and linear liquid alkanes. In some particular embodiments, the composition comprises one or a combination of coco-caprylate/caprate or C15-19 alkane.

The at least one emollient is present in a range from about 0.5% to about 40%, or from about 1% to about 39%, or from about 2% to about 38%, or from about 3% to about 37%, 4% to about 36.5%, 4% to about 36%, 4% to about 35%, 4% to about 35.5%, 4% to about 34%, 5% to about 35%, 5% to about 34%, 5% to about 33%, 5% to about 32%, 5% to about 31%, 5% to about 30%, 6% to about 37%, 7% to about 35%, 8% to about 37%, 9% to about 36%, 9% to about 35%, 10% to about 35%, or any suitable combination, sub-combination, range, or sub-range thereof by weight, based on the weight of the moisturizing peel cream. One of ordinary skill in the art, however, will appreciate that other ranges are within the scope of the invention.

Other suitable polar emollients may be selected from the group consisting of Isopropyl myristate, dicaprylyl ether, ethylhexyl palmitate, Isopropyl palmitate, cetearyl ethylhexanoate, Isononyl isononanoate, isopropyl isostearate, diisopropyl sebacate, coco caprylate/caprate, diisopropyl adipate, and combinations thereof.

Other suitable linear liquid alkanes (C11 to C20) may be selected from the group consisting of isohexadecane, C15-19 alkane, isododecane, undecane, tridecane, dodecane, and combinations thereof.

In some particular embodiments, the chemical peel component includes coco-caprylate/caprate, C15-19 alkane, dodecane and combinations thereof.

In some embodiments, the moisturizing peel cream includes Butyrospermum parkii butter, Theobroma cacao seed butter, Mangifera indica (mango) seed butter, Astrocaryum murumuru seed butter, Simmondsia chinensis (jojoba) butter caprylic/capric triglyceride, coco-caprylate/caprate, C12-15 alkyl benzoate, sunflower seed oil, olive oil, coconut oil, C15-19 alkane, C9-12 alkane, squalane, isopropyl myristate, and mixture thereof. In some particular embodiments, the moisturizing peel cream includes Butyrospermum parkii butter, Theobroma cacao seed butter, carnauba wax, Theobroma cacao grandiflorum seed butter, caprylic/capric triglycerides, coco-caprylate/caprate, Helianthus annuus (sunflower) seed oil, C15-19 alkane, dimethicone and combination thereof.

Thus, any one of or a combination of emollient is present, by weight, based on the total weight of the moisturizing peel cream, from about 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 to about 40 weight percent, including increments and ranges therein and there between.

In some embodiments, the moisturizing peel cream may further include at least one oil selected from the group consisting of Helianthus annuus (sunflower) seed oil, Sesamum indicum (sesame) seed oil, coconut oil, jojoba oil, isopropyl palmitate, isononyl isononanoate, apricot oil, dicaprylyl capric triglyceride, dicaprylyl carbonate, C12-15 alkyl benzoate, caprylic/capric triglyceride and combinations thereof.

More generally, in the various embodiments, the composition may include additional oil, which, when present, is generally immiscible in water, and may be selected from hydrocarbons, silicones, fatty alcohols, glycols and vegetable oils. The oil may include one or a combination of polar and non-polar oil. In some embodiments, the oil may be chosen from hydrocarbon-based oils from plants or of plant origin, mineral oil, ester oils, fatty alcohols containing from 12 to 26 carbon atoms, fatty acids containing from 12 to 26 carbon atoms and vinylpyrrolidone copolymers, and combinations thereof. In some particular embodiments, an oil may be chosen from purcellin oil (cetostearyl octanoate), squalane, hemisqualane, isononyl isononanoate, C12 to C15 alkyl benzoate, 2-ethylhexyl palmitate, isodecyl neopentanoate, tridecyl neopentanoate, octyldodecyl neopentanoate, 2-octyldodecyl stearate, 2-octyldodecyl erucate, oleyl erucate, isostearyl isostearate, 2-octyldodecyl benzoate, alcohol or polyalcohol octanoates, decanoates or ricinoleates, isopropyl myristate, isopropyl palmitate, butyl stearate, hexyl laurate, 2-ethylhexyl palmitate, 2-hexyldecyl laurate, 2-octyldecyl palmitate, 2-octyldodecyl myristate and 2-diethylhexyl succinate, cocoglyceride, cyclomethicone, dimethicone, dicaprylyl carbonate, dicapryl maleate, caprylic/capric triglyceride, isopropyl myristate, octyl stearate, isostearyl linoleate, lanolin oil, coconut oil, cocoa butter, olive oil, avocado oil, aloe extracts, jojoba oil, castor oil, fatty acid, oleic acid, stearic acid, fatty alcohol, cetyl alcohol, hexadecyl alcohol, diisopropyl adipate, hydroxybenzoate esters, benzoic acid esters of C9-C15 alcohols, alkanes, mineral oil, silicone, dimethyl polysiloxane, ether, polyoxypropylene butyl ether, polyoxypropylene cetyl ether, C12-C15 alkyl benzoate, aryl alkyl benzoate, Isopropyl Lauroyl sarcosinate, and any combinations thereof.

In accordance with the various embodiments, additional oil, when present, in the moisturizing peel cream is at present from about 1% to about 20%. One of ordinary skill in the art, however, will appreciate that other ranges are within the scope of the invention. The moisturizing peel cream may include more than one oil, such as a mixture of oils wherein one or the mixture of oils is present in an amount that alone or combined is present from about 1% to about 20% by weight based on the weight of the moisturizing peel cream, and wherein the total amount of additional oil is present up to about 20% of the moisturizing peel cream.

In accordance with some embodiments, the moisturizing peel cream may comprise at least one plant butter, the at least one butter selected from the group consisting of Butyrospermum parkii (shea) butter, Theobroma cacao (cocoa) seed butter, almond butter, aloe butter, apricot kernel butter, avocado butter, coconut cream butter, cupuacu butter, dhupu butter, hemp butter, jojoba butter, kokum butter, macadamia nut butter, mango butter and combinations thereof.

In some embodiments, the at least one butter has a melting end point in the range of from about 28 to about 55 degrees Celsius.

### Solvent

In accordance with the various embodiments, water is present in the moisturizing peel cream in a range from about 40% to about 80%, or from about 45% to about 75%, or from about 48% to about 70%, or any suitable combination, sub-combination, range, or sub-range thereof by weight, based on the weight of the moisturizing peel cream. One of ordinary skill in the art, however, will appreciate that other ranges are within the scope of the invention.

Thus, water is present, by weight, based on the total weight of the moisturizing peel cream, from about 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 to about 65 weight percent, including increments and ranges therein and there between.

The water used may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-saunier, water from Eaux Bonnes, water from Rochefort, water from Saint Christau, water from Les Fumades, water from Tercis-les-Bains or water from Avene. The water phase may also comprise reconstituted thermal water, that is to say a water comprising trace elements such as zinc, copper, magnesium, etc., reconstituting the characteristics of a thermal water.

The pH of the moisturizing peel cream is not limited but is generally not less than 3, and more particularly in a range from about 3 to about 6.3, or from about 3.5 to about 6, or about 3.8. The pH can be adjusted to the desired value by addition of a base (organic or inorganic) to the moisturizing peel cream, for example ammonia or a primary, secondary or tertiary (poly)amine, such as monoethanolamine, diethanolamine, triethanolamine, isopropanolamine or 1,3-propanediamine, or alternatively by addition of an inorganic or organic acid, advantageously a carboxylic acid, such as, for example, citric acid.

In some embodiments, the moisturizing peel cream includes one or more water based solvents selected from the group consisting of glycols, for example, glycerin and pentylene glycol, alcohols for solubilizing acid, for example, monoalcohols such as monohydric C1-C8 alcohols such as ethanol, propanol, butanol, isopropanol, isobutanol, and benzyl alcohol, and phenylethyl alcohol, and combinations thereof. In some embodiments, the water based solvents include glycerin and pentylene glycol. In some embodiments, glycerin is present in a range from about 5% to about 10%.

In accordance with the various embodiments, the amount of water based solvent present in the moisturizing peel cream is in the range from about 5% to about 10%, or from about 2% to about 9%, or from about 3% to about 8%, or any suitable combination, sub-combination, range, or sub-range thereof by weight, based on the weight of the moisturizing peel cream. One of ordinary skill in the art, however, will appreciate that other ranges are within the scope of the invention.

Thus, any one of or a combination of water based solvents may be present, by weight, based on the total weight of the moisturizing peel cream, from about 5, 5.5, 6, 6.5 7, 7.5, 8.5, 9.5 to about 10 weight percent, including increments and ranges therein and there between.

### Ratio of Emollient and Surfactant

The weight ratio of Emollient to Surfactant will vary but it typically be 1:1 to about 4:1. In various embodiments, the weight ratio of Emollient to Surfactant is about 1.1:1 to about 4:1, about 1.2:1 to about 4:1, about 1.3:1 to about 4:1, about 1.4:1 to about 4:1, about 1.5:1 to about 4:1, about 2:1 to about 4:1, about 2.5:1 to about 4:1, about 3:1 to about 4:1, about 1:1 to about 3.9:1, about 1:1 to about 3.8:1, about 1:1 to about 3.7:1, about 1:1 to about 3.6:1, about 1:1 to about 3.5:1, about 1:1 to about 3.0:1, including increments and ratio therein and there between.

This specific range of ratios has been found to be critical for achieving the desired properties of the final composition.

### Texturizer

In accordance with the various embodiments, the moisturizing peel cream may further includes at least one texturizer.

In some embodiments, the at least one texturizer is selected from the group consisting of silica, cellulose, boron nitride, mica, Hordeum vulgare seed flour, Aluminum Starch Octenylsuccinate and combination thereof.

In some embodiments, the at least one texturizer is present in a range from about 0 wt. % to about 2 wt. % based on the total weight of the composition. In various embodiments, the at least one texturizer is present from about 0.2 wt. % to about 1.8 or any suitable combination, sub-combination, range, or sub-range thereof by weight, based on the weight of the moisturizing peel cream. One of ordinary skill in the art, however, will appreciate that other ranges are within the scope of the invention.

Thus, each one of texturizers, alone or in combination, is present in an amount by weight, based on the total weight of the moisturizing peel cream, from about 0, 0.1, 0.2, 0.3, 0.4, 0.5, 0.7, 0.8, 0.9, 1.2, 1.4, 1.6, 1.8, or to about 2 weight percent, including increments and ranges therein and there between.

### Other Ingredients

In accordance with the various embodiments, the moisturizing peel cream may further include additional ingredients, generally including, but not limited to, chelating agents, pH adjusters, humectants, antioxidants, plant extracts, plant oils and butters, active components, essential oils, vitamins, antimicrobials and preservatives, fragrances, pearlescent agents, odor absorbers, coloring materials, fillers such as clays, talc, powders, including spherical/silica powders and mineral powders, penetrants, sequestrants, fragrances, dispersants, film-forming agents, ceramides, opacifiers, and combinations thereof. Non limiting examples of some of these optional additional ingredients are provided herein below.

### Chelating Agent

In some embodiments, one or more other components, such as chelating agents can be present in the moisturizing peel cream, in amounts from about 0.01% to about 2% by weight, from about 0.02% to about 1.5% by weight, from about 0.02% to about 1%, from about 0.02% to about 0.5%, and from about 0.025 to about 0.15%, or any suitable combination, sub-combination, range, or sub-range thereof by weight, based on the weight of the moisturizing peel cream or the moisturizing peel cream. In some exemplary embodiments, chelating agents are selected from ethylenediaminetetraacetic acid (EDTA), trisodium ethylenediamine disuccinate, tetrasodium glutamate diacetate, tetrasodium etidronate, tetrasodium pyrophosphate, pentasodium ethylenediamine tetramethylene phosphonate, sodium staminate and combinations of these.

Thus, one or a combination of chelating agents may be present, by weight, based on the total weight, each one or the combination present from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0 up to about 2 weight percent, including increments and ranges therein and there between.

### Optional Actives and Other Ingredients

In some embodiments, there may be one or more additives present in the moisturizing peel cream the additive selected from, for example, humectants, such as acetamide MEA, glycols, such as glycerin and propylene glycol, alcohol, anti-microbial components, salicylic acid, alpha acid, antioxidants, including, but not limited to, phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin (Scutellaria Baicalensis root extract), pine bark extract (Pinus Pinaster bark/bud extract), ellagic acid, hyaluronic acid and its derivatives, bisabolol, dipotassium glycyrrhizate, glycyrrhizic acid, and vitamins and vitamin derivatives, such as tocopherol and ascorbic acid, and combinations thereof.

In some embodiments, there may be one or more other additives present in the moisturizing peel cream, the other components selected from the group consisting of fillers such as clays, talc, powders, including spherical/silica powders and mineral powders, penetrants, sequestrants, fragrances, dispersants, film-forming agents, ceramides, opacifiers and combinations thereof.

In some embodiments, there may be one or more other components present in the moisturizing peel cream, according to the disclosure, the other components selected from sodium hydroxide, disodium EDTA, sodium citrate, sodium hyaluronate, capryloyl salicylic acid, lactic acid, methyl dihydro jasmonate, acetyl trifluoromethyl phenyl valyglycine, pentaerythrityl tetra-di-t-butyl hydroxydrocinnamate, n-hydroxysuccinimide, palmitoyl oligopeptide, chrysin, palmitoyl tetrapeptide-7, yeast extract, citric acid and combinations thereof.

In some embodiments, additives may include one or a combination of additives selected from the group consisting of sodium hydroxide, trisodium ethylenediamine disuccinate, tocopherol, hydrogen peroxide, and combinations thereof.

In some embodiments, additives may include one or a combination of antimicrobial agents and their salts, selected from, for example, chlorphenesin, caprylyl glycol, phenoxyethanol, caprylhydroxamic acid, benzoic acid, salicylic acid, benzyl alcohol, phenethyl alcohol, benzalkonium chloride, 4-hydroxyacetophenone, piroctone olamine, hexyl glycerin, ethylhexylglycerin, octylglycerin, benzylglycerin, 3-heptoyl-2,2-propandiol, and 1,2-hexandiol, and pentylene glycol, and combinations thereof.

In some particular embodiments, one or more additives in any of the components are selected from the group consisting of sodium hydroxide, trisodium ethylenediamine disuccinate, tocopherol, hydrogen peroxide, chlorophenesin, salicylic acid, phenoxyethanol, bisabolol, dipotassium glycyrrhizate, glycyrrhizic acid, sodium hydroxide, trisodium ethylenediamine disuccinate, tocopherol, sodium hyaluronate, propanediol, hydrogen peroxide, and combinations thereof.

In some embodiments, the moisturizing peel cream is essentially free, or free, or devoid of one or more ingredients selected from the group consisting of silicones, including silicone oils, hydrogen peroxide and its analogs, sulfates, and combinations thereof.

Although the aforementioned optional components are given as an example, it will be appreciated that other optional components compatible with cosmetic applications known in the art may be used.

In accordance with the various embodiments, the number of additives present in the moisturizing peel cream can range from about 0 to about 50%, from about 0.5 to about 30%, from about 1.5 to about 20%, and from about 5 to about 15%, or any suitable combination, sub-combination, range, or sub-range thereof by weight, based on the weight of the moisturizing peel cream or the moisturizing peel cream.

In some embodiments, one or more additives, alone or in combination, can be present in one or both of the moisturizing peel cream and the moisturizing peel cream from about 0.05 to about 50% by weight, from about 0.05% to about 2.5% by weight, from about 0.1 to about 2%, from about 0.25 to about 1.5%, and from about 0.5 to about 1.25%, or any suitable combination, sub-combination, range, or sub-range thereof by weight, based on the weight of the moisturizing peel cream or the moisturizing peel cream.

In some embodiments, one or more other additives, such as preservatives, vitamins, preservatives, and the like, alone or in combination, can be present in one or both of the moisturizing peel cream and the moisturizing peel cream from about 0.05 to about 50% by weight, from about 0.05% to about 25% by weight, from about 0.1 to about 10%, from about 0.25% to about 5%, and from about 0.5 to about 3.5%, or any suitable combination, sub-combination, range, or sub-range thereof by weight, based on the weight of the moisturizing peel cream or the moisturizing peel cream.

Thus, one or a combination of optional additives may be present in the moisturizing peel cream, by weight, based on the weight of the moisturizing peel cream, each one or the combination present from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0 0.80, 0.90, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 to about 50 weight percent, including increments and ranges therein and there between.

The instant disclosure also relates to methods of using the compositions described herein. For example, the compositions can be used in a method that comprises applying the cosmetic compositions to the skin of humans. In some cases, the composition is applied to the face.

### EXAMPLES

### Example 1: Inventive (INV) and Comparative (COMP) Examples

TABLE 1

| | INV1 | INV2 | INV3 | INV4 | INV5 | INV6 | INV7 | COMP1 | COMP2 | COMP3 | COMP4 | COMP5 |
|---|---|---|---|---|---|---|---|---|---|---|---|---|
| INCI Name | Concentration of the Ingredient | | | | | | | | | | | |
| Total Water + Water | 58.46 | 58.31 | 57.25 | 57.31 | 59.46 | 71.79 | 44.97 | 32.31 | 59.81 | 62.11 | 73.95 | 58.99 |
| Phase Solvent | | | | | | | | | | | | |
| WATER | 51.46 | 51.31 | 50.25 | 50.31 | 52.46 | 64.79 | 39.59 | 25.31 | 52.81 | 55.11 | 66.95 | 51.93 |
| GLYCERIN | 7 | 7 | 7 | 7 | 7 | 7.00 | 5.38 | 7 | 7 | 7 | 7 | 7.05 |
| Total Hydroxy Acids | 10.45 | 10.45 | 10.45 | 10.45 | 10.45 | 10.45 | 8.04 | 10.45 | 10.45 | 10.45 | 10.45 | 10.57 |
| SALICYLIC ACID | 0.45 | 0.45 | 0.45 | 0.45 | 0.45 | 0.45 | 0.35 | 0.45 | 0.45 | 0.45 | 0.45 | 0.45 |
| GLUCONOLACTONE | 4 | 4 | 4 | 4 | 4 | 4.00 | 3.08 | 4 | 4 | 4 | 4 | 4.04 |
| GLYCOLIC ACID | 6 | 6 | 6 | 6 | 6 | 6.00 | 4.62 | 6 | 6 | 6 | 6 | 6.07 |
| Total Polymer | 0.35 | 0.2 | 0.2 | 0.2 | 0.35 | 0.42 | 0.27 | 0 | 0 | 4.2 | 0.3 | 0.48 |
| Thickener | | | | | | | | | | | | |
| XANTHAN GUM | 0.1 | 0.2 | 0.2 | 0.2 | 0.1 | 0.12 | 0.08 | 0 | 0 | 0.2 | 0.1 | 0.14 |
| CETYL | 0.25 | 0 | 0 | 0 | 0.25 | 0.30 | 0.19 | 0 | 0 | 0 | 0.2 | 0.35 |
| HYDROXYETHYL | | | | | | | | | | | | |
| CELLULOSE | | | | | | | | | | | | |
| HYDROXYETHYL | 0 | 0 | 0 | 0 | 0 | 0 | 0 | 0 | 0 | 4 | 0 | 0 |
| ACRYLATE/SODIUM | | | | | | | | | | | | |
| ACRYLOYLDIMETHYL | | | | | | | | | | | | |
| TAURATE | | | | | | | | | | | | |
| COPOLYMER | | | | | | | | | | | | |
| Total Filler | 0 | 0 | 1 | 1 | 0 | 0 | 0 | 0 | 0 | 2 | 0 | 0 |
| SILICA | 0 | 0 | 1 | 1 | 0 | 0 | 0 | 0 | 0 | 0 | 0 | 0 |
| ALUMINUM | | | | | | | | | | | | |
| STARCH | 0 | 0 | 0 | 0 | 0 | 0 | 0 | 0 | 0 | 2 | 0 | 0 |
| OCTENYLSUCCINATE | | | | | | | | | | | | |
| Total Surfactants + | 27.50 | 27.5 | 27.5 | 27.5 | 27.5 | 15.1 | 44.2 | 55 | 27.5 | 17.5 | 12.00 | 26.6 |
| Emollients | | | | | | | | | | | | |
| Total Surfactants | 11.50 | 11.50 | 11.50 | 11.50 | 11.50 | 6.30 | 8.85 | 23.00 | 11.50 | 6.50 | 5.00 | 11.14 |
| PEG-100 | 2 | 2 | 2 | 2 | 2 | 1.10 | 1.54 | 4 | 2 | 1 | 0.5 | 1.94 |
| STEARATE | | | | | | | | | | | | |
| POLYSORBATE | 0.5 | 0.5 | 0.5 | 0.5 | 0.5 | 0.25 | 0.38 | 1 | 0.5 | 0.5 | 0.5 | 0.48 |
| 20 | | | | | | | | | | | | |
| CETEARYL | 2.84 | 2.84 | 2.84 | 2.84 | 2.84 | 1.56 | 2.18 | 5.68 | 2.84 | 0.71 | 0.71 | 2.75 |
| ALCOHOL | | | | | | | | | | | | |
| CETEARETH-20 | 1.16 | 1.16 | 1.16 | 1.16 | 1.16 | 0.64 | 0.89 | 2.32 | 1.16 | 0.29 | 0.29 | 1.13 |
| GLYCERYL | 5 | 5 | 5 | 5 | 5 | 2.75 | 3.85 | 10 | 5 | 3 | 2 | 4.84 |
| STEARATE | | | | | | | | | | | | |
| LAURETH-4 | 0 | 0 | 0 | 0 | 0 | 0 | 0 | 0 | 0 | 0 | 0 | 0 |
| STEARIC ACID | 0 | 0 | 0 | 0 | 0 | 0 | 0 | 0 | 0 | 1 | 1 | 0 |
| Total Emollients | 16 | 16 | 16 | 16 | 16 | 8.8 | 35.4 | 32 | 16 | 11 | 7 | 15.50 |
| CARNAUBA WAX | 0 | 0 | 0 | 0 | 0 | 0 | 0 | 0 | 0 | 0 | 0 | 0 |
| THEOBROMA | 0 | 0 | 0 | 0 | 0 | 0 | 0 | 0 | 0 | 3 | 0 | 0 |
| CACAO SEED | | | | | | | | | | | | |
| BUTTER | | | | | | | | | | | | |
| THEOBROMA | 4 | 4 | 4 | 0 | 8 | 2.20 | 3.08 | 8 | 4 | 0 | 3 | 3.88 |
| GRANDIFLORUM | | | | | | | | | | | | |
| SEED BUTTER | | | | | | | | | | | | |
| BUTYROSPERMUM | 4 | 4 | 4 | 0 | 8 | 2.20 | 3.08 | 8 | 4 | 2 | 1 | 3.88 |
| PARKII | | | | | | | | | | | | |
| (SHEA) BUTTER | | | | | | | | | | | | |
| CAPRYLIC/CAPRIC | 0 | 0 | 0 | 0 | 0 | 0 | 23.08 | 0 | 0 | 0 | 0 | 0 |
| TRIGLYCERIDE | | | | | | | | | | | | |
| COCO- | 1 | 2 | 1 | 9 | 0 | 0.55 | 0.77 | 6 | 3 | 1 | 1 | 0.97 |
| CAPRYLATE/ | | | | | | | | | | | | |
| CAPRATE | | | | | | | | | | | | |
| HELIANTHUS | 0 | 0 | 0 | 0 | 0 | 0 | 0.00 | 0 | 0 | 5 | 0 | 0 |
| ANNUUS SEED | | | | | | | | | | | | |
| OIL | | | | | | | | | | | | |
| C15-19 ALKANE | 5 | 5 | 5 | 5 | 0 | 2.75 | 3.85 | 10 | 5 | 0 | 1 | 4.84 |
| DIMETHICONE | 2 | 1 | 2 | 2 | 0 | 1.10 | 1.54 | 0 | 0 | 0 | 1 | 1.94 |
| Other ingredients | 3.24 | 3.54 | 3.54 | 3.54 | 2.24 | 2.24 | 2.49 | 2.24 | 2.24 | 3.74 | 3.30 | 3.32 |

### Example 3: Evaluation of the Performance of the Compositions

The viscosity was determined using an oscillation amplitude rheology experiment, measuring rheological properties within a shear strain of 0.001%-100% at 10 rad/s and 25° C. The viscosity value presented was the complex viscosity at 0.01% strain, which was in the linear viscoelastic region for all samples. To “pass” the viscosity performance criteria (i.e., composition was considered a thick cream), the viscosity of the composition must exceed 200 Pa·s.

The results of Inventive and Comparative Examples are shown respectively in Table 2 and Table 3 below.

TABLE 2
Performance Evaluation of Inventive Examples

| | Anti-Pilling | Stability | Viscosity |
|---|---|---|---|
| Composition | Performance | Performance | (Pa · s) |
| INV1 | Pass | Pass | 1778 |
| INV2 | Pass | Pass | 2139 |
| INV3 | Pass | Pass | 2915 |
| INV4 | Pass | Pass | 1860 |
| INV5 | Pass | Pass | 2566 |
| INV6 | Pass | Pass | 292 |
| INV7 | Pass | Pass | 286 |

TABLE 3
Performance Evaluation of Comparative Examples

| | Anti-Pilling | Stability | Viscosity | |
|---|---|---|---|---|
| Composition | Performance | Performance | (Pa · s) | Results |
| COMP1 | Not | Fail | Not | Composition was above the permissible |
| | tested | | tested | range for total emollients + surfactants; |
| | | | | therefore, it failed the stability criteria |
| | | | | (instantaneous phase separation at 25 C.). |
| COMP2 | Pass | Fail | 977 | Composition was below the permissible |
| | | | | range for polymer thickener; therefore, it |
| | | | | failed the stability criteria (phase |
| | | | | separation at 45 C.). |
| COMP3 | Fail | Pass | 478 | Composition was far above the |
| | | | | permissible range of polymer thickener; |
| | | | | therefore, it failed the pilling criteria. |
| COMP4 | Fail | Pass | 143 | Composition was below the permissible |
| | | | | range for total emollients + surfactants; |
| | | | | therefore, it failed the viscosity and pilling |
| | | | | criteria. |
| COMP5 | Fail | Pass | 1280 | Composition was above the permissible |
| | | | | range for polymer thickener; therefore, it |
| | | | | failed the pilling criteria. |

To evaluate “anti-pilling performance,” a fixed volume of formula was applied in an even layer on a fixed surface area of clean, dry skin. After allowing the formula to absorb into the skin, the skin was then subjected to a friction gesture wherein clean fingers were repeatedly swiped across the skin with a fixed pressure. If aggregates of formula (“noodles” or “pills”) were produced during this friction gesture, the composition was said to fail the anti-pilling performance criteria. If fingers were able to repeatedly rub across the film left on skin by the formula without producing noodles/pills, the composition was said to pass the anti-pilling performance criteria.

According to the Piling Evaluation in Table 2 and 3, the Inventive and Comparatives Examples exhibited different behavior after being submitted to a simple application on the skin and to a friction gesture.

The Comparative Examples contained hydroxy acids that generated a high electrolyte content and consequently required a high amount of polymer thickeners to give a thick and rich texture. After being evaluated for piling, some Comparative Examples exhibited low level of piling after being applied on the skin but did exhibit a high level of piling after the friction gesture (i.e. Comp 1, Comp2, Comp3, Comp5).

On the contrary, the Inventive Examples demonstrated a low piling in both cases with and without friction gesture. They surprisingly showed a great resistance to piling by employing a specific thickening mechanism that relied on a high surfactant/co-surfactant load (i.e. 8%). This system allowed to minimize the amount of polymer thickeners. It was surprising that the total amount of polymer thickeners needed to be less than 3 wt. % to have less piling and still be able to have a very thick, creamy and luxurious texture. These results showed that the lower total amount of polymer thickener was necessary to have both the thick, creamy, luxurious texture and the non piling effect.

### Example 4: Stability of the Inventive Compositions

The Inventive Compositions were submitted to various temperatures for 2 months. They were considered stable as they didn't show any phase separation with time and exposure to elevate temperature. The results are reported below in Table 4.

TABLE 4

| | Day 1 | | | |
|---|---|---|---|---|
| Inventive | @Room | 2 months | 2 months | 2 months |
| Example 2 | Temp. | @4 C. | @25 C. | @45 C. |
| Observa- | Formula | Formula | Formula | Formula |
| tion | showed no | showed no | showed no | showed no |
| | phase | phase | phase | phase |
| | separation | separation | separation | separation |
| | or creaming | or creaming | or creaming | or creaming |
| | Oil droplets | Oil droplets | Oil droplets | Oil droplets |
| | size | size | size | size |
| | remained | remained | remained | remained |
| | stable | stable | stable | stable |

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one,” as used herein, means one or more and thus includes individual components as well as mixtures/combinations.

The transitional terms comprising,” “consisting essentially of” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.”

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A moisturizing cream peel composition comprising:
from up to about 20 wt. % of at least one hydroxy acid;
from about at least 6 wt. % of at least one surfactant;
at least 0.2% of one polymer thickener;
at least one emollient;
at least one water-based solvent;
water;
wherein the ratio between (emollient):(surfactant) is from about 1:1 to about 4:1;
wherein the composition has a complex viscosity above 200 Pa·S, is stable, resistant to pilling and has a pH is of about 3.5 to about 6.3; and
wherein the weight percentages are based on the total weight of the composition.

2. The composition according to claim 1, wherein the at least one hydroxy acid is chosen from alpha-, poly-, beta-hydroxy acids, and combinations thereof.

3. The composition according to claim 1, wherein the alpha hydroxy acid, if present, is less than about 8 wt. % based on the total weight of composition.

4. The composition according to claim 1, wherein the alpha-hydroxy acid is chosen from glycolic acid, lactic acid, tartaric acid, mandelic acid, citric acid, glycolic citric acid, mandelic acid, tartaric acid, kojic acid, malic acid, ester derivatives thereof and combinations thereof.

5. The composition according to claim 1, wherein the beta-hydroxy acid is chosen from salicylic acid, betaine salicylate, tropic acid and mixture thereof.

6. The composition according to claim 1, wherein the poly-hydroxy acid is chosen from gluconolactone, lactobionic acid, galactose, and mixture thereof.

7. The composition according to claim 1, wherein the composition is free of beta-hydroxy acid.

8. The composition according to claim 1, wherein the composition is free of poly hydroxy acid.

9. The composition according to claim 1, further comprising at least one additional hydroxy acid chosen from alpha-, poly-, and beta-hydroxy acids, and combinations thereof.

10. The composition according to claim 1, wherein the at least one surfactant is chosen from fatty alcohol, cetearyl alcohol, stearic acid, polysorbate 20, ceteareth-20, PEG-100 stearate, polyglyceryl-10 stearate, glyceryl stearate, sorbitan stearate, and combinations thereof.

11. The composition according to claim 1, wherein the at least one surfactant is present from about 6.1 to about 15 wt. % based on the total weight of the composition.

12. The composition according to claim 1, wherein the at least one polymer thickener is selected from the group consisting of xanthan gum, sclerotium gum, hydroxypropyl starch phosphate, cetyl hydroxyethyl cellulose, carbohydrate based gellifying agents, organic thickeners and mixture thereof.

13. The composition according to claim 1, wherein the amount of polymer thickeners is up to about 0.45 wt. % based on the total weight of the composition.

14. The composition according to claim 1, wherein the at least one emollient is chosen from plant butters, oils, and combinations thereof.

15. The composition according to claim 15, wherein the at least emollient is chosen from Butyrospermum parkii butter, Theobroma cacao seed butter, Mangifera indica (mango) seed butter, Astrocaryum murumuru seed butter, Simmondsia chinensis (jojoba) butter caprylic/capric triglyceride, coco-caprylate/caprate, C12-15 alkyl benzoate, sunflower seed oil, olive oil, coconut oil, C15-19 alkane, C9-12 alkane, squalane, isopropyl myristate, and mixture thereof.

16. The composition according to claim 1, wherein the at least one water-based solvents is chosen from glycerin, propylene glycol, propanediol, butylene glycol, pentylene glycol, sorbitol, xylitol and mixture thereof.

17. The composition according to claim 1, wherein the water is present from about 30 wt. % to about 80 wt. % based on the total weight of the composition.

18. The composition according to claim 1 wherein the complex viscosity is above 200 Pa·S.

19. The composition according to claim 1, further comprising at least one texturizer.

20. A method for treating skin comprising applying the cosmetic composition of claim 1 to the skin.
